(19) United States
(12) Patent Application Publication
(10) Pub. No.: US 2007/0049885 A1
(43) Pub. Date: Mar. 1, 2007

(54) Potty training device

(76) Inventor: Wanda Phillips, Anthony, FL (US)

Correspondence Address: Leander F. Aulisio, 320 23rd Street # 705, Arlington, VA, 22202, US

(21) Appl. No.: 11/359,559

(22) Filed: Feb. 23, 2006

Related U.S. Application Data
(60) Provisional application No. 60/712,901, filed on Sep. 1, 2005.

(51) Int. Cl. A61F 13/15

(52) U.S. Cl. 604/361

(57) ABSTRACT

A potty training method and device comprising a training pant, a timing device having an alarm mechanism, and a compartment for holding said timing device; the alarm mechanism capable of being set to correspond to the drinking capacity of a child.

The present non-provisional application is based on provisional application No. 60/712,901 filed on Sep. 1, 2005.

### BACKGROUND OF THE INVENTION

1. Field of the Invention

The present invention relates generally to a potty training method and a device comprising a training pant, a timing device having an alarm mechanism, and a compartment for holding the timing device. The ease of operation of the potty training device encourages a child to use a toilet independently.

2. Discussion

Potty training continues to present a challenge for both parent and child. Particularly challenging aspects of potty training involve conditioning the child to recognize the sensation of needing to use the toilet and establishing a routine for regular toilet visits. The prior art discloses a variety of devices that have been developed to aid parents and children in this task. One such device is a training pant that a child can raise and lower without assistance. U.S. Patent Application Publication No. 2004/0243092 to Karami (Dec. 2, 2004) relates to a pull-up type diaper that contains front and back waist regions and side seals. The side seals can be secured together when the diaper is in assembled ready-to-wear condition.

Devices that contain timing and alarm functions have also been disclosed. For example, U.S. Pat. No. 5,365,496 issued on Nov. 15, 1994, to Tolan-Samilow, describes a device for conditioning children to maintain a schedule and to perform tasks such as potty training. The device comprises an alarm circuit and timer that can be set for adjustable alarm intervals. In one embodiment, the device has a housing that resembles a children's character. In another embodiment, the device has a fastener in the form of a wrist band, a pendant or a belt, capable of being fastened to a user.

U.S. Patent Publication No. 2003/0123330 to Carter, et al. describes an event timer having an adaptable interval for potty training children. The timer has an input function with at least one button and an output for signaling the expiration of a timing interval by audio and/or visual means. The event timer can take the form of a clock, watch, necklace, belt, doll, animal or vehicle.

U.S. Patent Publication No. 2002/0009018 Patella describes a device for conditioning children having a central processing unit within an elliptical housing, a timer and a signaling means. The signaling means is activated at the end of a pre-selected time interval and consists of an audible, visual or vibratory signal or combination thereof.

U.S. Patent Application Publication No. 2005/0137542 to Underhill et al. discloses a training pant having attached thereon an electrochromic display. The display includes an animated cartoon character or a moving vehicle. The display can also create symbols or words. A power source, circuitry, and sensors can be combined with the electrochromic display to serve as an indicator when the child has urinated or has had a bowel movement. In another embodiment, the electroactive display can be combined with a timing circuit. The display can then indicate the time to replace a disposable diaper or the like.

None of the above inventions and patents, taken alone or in combination, is seen to describe the present invention as claimed.

### SUMMARY OF THE INVENTION

This invention relates to a potty training device for use in training a child to use a toilet. The device of the present invention comprises a disposable training pant having a timing device and a compartment for housing said timing device. In a preferred embodiment, the training pant comprises a “pull up” type diaper that can be removed by a child without the assistance of an adult, thus enabling the child to use a potty independently.

The timing device contains an alarm mechanism capable of producing a signal in order to alert a child to use or attempt to use the potty. In one embodiment, the alarm mechanism can be set for a 15 minute interval when the child is considered to be a heavy drinker, i.e., one who consumes an above-average quantity of fluids and experiences more frequent urination. For a child who is considered to be a medium drinker, i.e., one who consumes an average quantity of fluids, the alarm can be set for a 25 minute interval. For a child who is considered to be a light drinker, i.e., one who consumes less than an average amount of fluids and experiences less frequent urination, the alarm can be set for a 35 minute interval.

In an alternative embodiment, the training device of the present invention can be employed to assist the child in managing bowel movements.

The training pant also comprises a compartment for holding the timing device. The compartment cart be integral with the training pant as by sewing onto the pant. Alternatively, the compartment can be affixed to the pant with adhesive material. When an adhesive material is employed, the compartment can be pressed onto the surface of the pant. It is within the scope of the present invention to employ a removable adhesive material.

### BRIEF DESCRIPTION OF THE DRAWINGS

The accompanying drawing, in conjunction with the general description given above, and the detailed description of the preferred embodiments given below, serves to illustrate and explain the principles of the preferred embodiments of the best mode of the invention presently contemplated, wherein:

FIG. 1 is a perspective view of a preferred embodiment of a potty training device according to the present invention.

### DETAILED DESCRIPTION OF THE INVENTION

In the following detailed description of the invention, reference is made to the accompanying drawing which forms a part of the disclosure, and, in which are shown by way of illustration, and not of limitation, specific embodiments by which the invention may be practiced. The embodiments illustrated are described in sufficient detail to enable those skilled in the art to practice the invention. Other embodiments may be utilized and derived therefrom, such that structural and logical substitutions and changes may be made without departing from the scope of the invention. The following detailed description, therefore, is not to be taken in a limiting sense, and the scope of the invention is defined only by the appended claims, along with the full range of equivalents to which such claims are entitled.

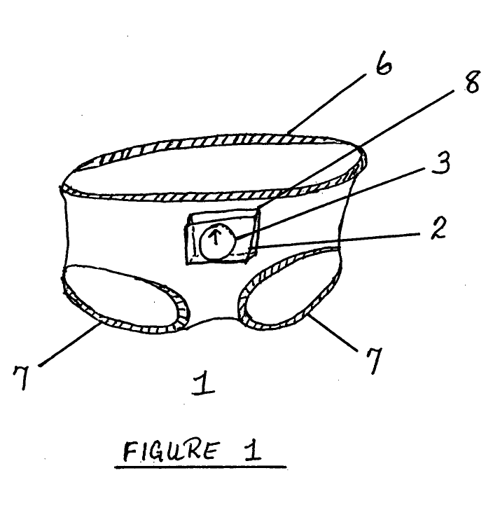

FIG. 1. shows a training pant [1] that can be raised or lowered by a child without parental assistance. It can have an elastomeric waist [6] to facilitate easy raising and lowering of the pant by the child during bathroom visits. Optionally, the training pant [1] can also have elastomeric leg bands [7]. In a preferred embodiment, the training pant [1] is a disposable “pull-up” type diaper. The training pant [1] can be made from a woven material, a non-woven material or mixtures thereof.

The training pant [1] has a compartment [2] for holding a timing device [3] having an alarm mechanism. In one embodiment, the compartment [2] has a front surface, a back surface and an opening [8] for insertion of a timing device [3]. In another embodiment, the compartment [2] is sealed on all sides. The opening [8] can be optionally resealable.

The compartment [2] can be integral with the training pant [1]. In such a case, the compartment can be sewn onto the pant. In an alternative embodiment, the compartment [2] can be removably affixed to the training pant [1]. The compartment can be affixed to any location on the pant. The alarm signal serves to remind the child that it is time to attempt to use the potty. The signal that is produced can be audible, visual, vibratory, or any combination thereof.

The timing device [3] can be either mechanical, e.g., wind-up, or electronic, e.g., battery operated, or a combination thereof. In a preferred embodiment, the timing device comprises a clock face. The clock face can be any shape, although a round clock face is preferred. The time can be displayed in digital format for an electronic device or at least one movable clock hand for a mechanical device.

The method of the invention comprises the steps of (a) obtaining a device of this invention, (b) placing a disposable training pant on a child who is being potty trained, (c) selecting a time-interval suited to the drinking capacity of a child and (d) setting the alarm mechanism to correspond to the selected time-interval.

In one embodiment, the alarm on the timing device is pre-set for a single specified time interval. The device can have a specified time interval that can range from 5 minutes to 60 minutes, or longer. Parents can select an alarm interval that corresponds to the drinking capacity of their individual child. For example, parents whose child consumes a large quantity of fluids and thus urinates more frequently, i.e., a “heavy drinker,” can select a device having a 15 minute alarm interval. In this example, the alarm activates every 15 minutes conditioning the child to use or attempt to use the potty every 15 minutes in order to reduce or avoid an “accident.” In one embodiment, the timing device has a button that can be pressed to set the alarm for the timed interval. The timer can be engaged by pressing a button on the timing device which sets the timer in motion. Once the alarm has triggered, the timer can be reset for another 15 minutes by again pressing the button on the timing device.

Parents whose child consumes an average quantity of fluids, i.e., a “medium drinker,” can select a 25 minute alarm interval. Parents whose child consumes a less than average quantity of fluids, i.e., a “light drinker,” can select a 35 minute pre-set alarm interval.

In a preferred embodiment, the timing device contains an alarm mechanism having at least three pre-set timed intervals that can be used for children who are heavy drinkers, medium drinkers or light drinkers. In this embodiment, the timing device contains three buttons; the three buttons corresponding to pre-determined timing intervals for a child who is considered to be a “heavy drinker,” a medium drinker,” and a “light drinker.” For example, one button can be pre-set for a 15 minute timed interval for use in conjunction with a child who is considered to be a “heavy drinker,” a second button can be pre-set for a 25 minute timed interval for use in conjunction with a child who is considered to be a “medium drinker,” and a third button can be pre-set for a 35 minute timed interval for use in conjunction with a child who is considered to be a “light drinker.”

In another embodiment, the timing device comprises a knob. The timer can be set by moving the knob to the time desired for the alarm to activate. In yet another embodiment, the timing device comprises at least one clock hand that can be operated in a manner similar to the knob to set the timer for the desired timed interval.

When the method is used to assist a child in managing bowel movements, the timer can be set to activate the alarm about 30-60 minutes after the child has eaten a meal. These time intervals are provided as approximations and can be adjusted according to the toilet habits of an individual child.

The device of this invention comprises a training pant, a timing device having an alarm mechanism, and a compartment for holding the timing device. In a preferred embodiment, the training pant is a disposable training pant. More preferably, the disposable training pant is a diaper. The diaper preferably has an elastomeric waist for easy removal by a child. Most preferably, the diaper contains both an elastomeric waist and two elastomeric leg bands.

The diaper is prepared from a material selected from the group consisting of a woven material, a non-woven material, and mixtures thereof. In a preferred embodiment, the diaper is prepared from cellulosic fiber and superabsorbent polymer (SAP) particles. Other ingredients such as bactericides and the like can be present in the diaper material.

The compartment for holding the timing device has a front surface and a back surface. The front surface is attached to the back surface on all sides except one that functions as an opening for insertion of the timing device. Preferably, the compartment has a rectangular shape or a square shape, although other shapes and designs are within the scope of the invention.

The opening of the compartment is preferably re-sealable so that the timing device is not in danger of falling out of said compartment. The opening can be sealed with a Velcro® closure, a Zip-Lock® closure, an electrostatic closure or the like.

The front surface and the back surface of the compartment is preferably prepared from a plastic material. In a preferred embodiment, the plastic material comprises a transparent flexible synthetic polymer. The timing device can also be manufactured from a flexible synthetic polymer to increase the comfort level for the child. In an alternative embodiment, the training device of the present invention can be employed to assist a child in managing bowel movements.

It is within the scope of the present invention to permanently seal all sides of the compartment after the timing device is inserted into the compartment. In one embodiment, the timing device itself can be prepared from disposable material so that the entire diaper and device combination is disposable.

Preferably, the compartment that contains the timing device is attached to the training pant by being pressed onto an outer surface of the pant. When the training pant is ready for replacement, the compartment can be removed from the soiled pant and pressed onto a fresh training pant. In this manner, one compartment and one timing device can be employed for repeated use with a multitude of fresh training pants

While the invention has been described in various embodiments above, there is no intent to limit the scope of the present invention except as recited in the claims that follow.

I claim:

1) A device for potty training a child comprising (a) a training pant, (b) a timing device having an alarm mechanism, and
(c) a compartment for holding the timing device.

2) A device according to claim 1 wherein the training pant is a disposable diaper.

3) A device according to claim 1, wherein training pant comprises an elastomeric waist.

4) A device according to claim 3, wherein the training pant further comprises two elastomeric leg bands.

5) A device according to claim 1, wherein the compartment for holding the timinig device is comprised of a front surface, a back surface, and an opening for insertion of the timing device.

6) A device according to claim 5, wherein the opening comprises a resealable opening.

7) A device according to claim 1, wherein the compartment for holding a timing device is sealed on all sides.

8) A device according to claim 1, wherein the compartment is removably attached to the training pant.

9) A device according to claim 8, wherein the compartment is attached to the training pant by being pressed onto an outer surface of the training pant.

10) A device according to claim 1, wherein the compartment comprises a transparent material.

11) A device according to claim 1 wherein the timing device is selected from the group consisting of a mechanical timing device and a digital timing device.

12) A device according to claim 11 wherein the mechanical timing device additionally comprises a clock face with at least one moveable clock hand.

13) A device according to claim 12 wherein the alarm mechanism is operated by setting the clock hand to a selected timed interval.

14) A device according to claim 11 wherein the timing device further comprises at least one button which corresponds to a pre-set alarm timed interval.

15) A device according to claim 14 wherein at least one button is located around the perimeter of the timing device.

16) A device according to claim 14 wherein the timing device comprises three buttons which correspond to three pre-set alarm time intervals selected according to the drinking capacity of a child.

17) A device according to claim 16 wherein the three pre-set alarm time intervals correspond to a child who is a heavy drinker, a child who is a medium drinker, and a child who is a light drinker.

18) A device according to claim 1 wherein the alarm mechanism produces a signal selected from the group consisting of sound, motion light, strobe light, vibration, olfactory, and combinations thereof.

19) A method of training a child to use a potty comprising the steps of:
(1) placing a training device on a child,
(2) selecting a timed interval based on the drinking capacity of the child, and
(3) setting the alarm to activate at the selected timed interval, wherein the training device comprises a training pant, a timing device having an alarm mechanism, and a compartment for holding the timing device.

20) A method of training a child to use a potty comprising the steps of:
(1) placing a training device on a child,
(2) selecting a timed interval based on the eating capacity of the child, and
(3) setting the alarm to activate at the selected time interval, wherein the training device comprises a training pant, a timing device having an alarm mechanism, and a compartment for holding the timing device.
